(19) United States
(12) Patent Application Publication
(10) Pub. No.: US 2024/0100282 A1
(43) Pub. Date: Mar. 28, 2024

(54) THERAPY SYSTEM FOR RESPIRATORY-RELATED DISORDERS, AND PATIENT INTERFACE AND HEADGEAR FOR USE IN SAME

(71) Applicant: RESMED PTY LTD, Bella Vista (AU)

(72) Inventor: Kenneth John TAYLOR, Sydney (AU)

(21) Appl. No.: 18/476,024

(22) Filed: Sep. 27, 2023

(30) Foreign Application Priority Data
Sep. 28, 2022 (AU) 2022902804

(51) Int. Cl. A61M 16/06; A61M 16/08

(52) U.S. Cl. CPC A61M 16/0672; A61M 16/0875; A61M 2210/0618

(57) ABSTRACT

The invention relates to a respirator therapy system including a respiratory therapy device, a patient interface and an air circuit delivering pressurised air to the patient interface. The air circuit is formed from two or more lengths of tubing connected to each other by a connecting mechanism. The connecting mechanism is configured such that the respective connectors are both fluidly and electrically connected.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist and include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is abroad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth. Other masks may be uncomfortable or impractical for the present technology if they require a patient to insert a portion of a mask structure in their mouth to create and maintain a seal via their lips. Some masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact on the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310: WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

It will be appreciated that the patient has a number of options available to them for the patient interface and seal-forming structure. However, it may be a significant investment for the patient to switch between these options, particularly since they may be designed to be worn with specifically designed positioning and stabilising structures.

### 1.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique in widespread usage for positioning the seal-forming structure on the patient's face is the provision of one or more straps and/or stabilising harnesses as the positioning and stabilising structure. These structures are often referred to as headgear.

The headgear can include a plurality of straps and buckles or the like which engage with the patient interface. Some examples of headgear suffer from being one or more of ill-fitting, bulky, uncomfortable and awkward to use.

### 1.2.3.1.3 Pressurised Air Conduit

In one type of treatment system, a flow of pressurised air is provided to a patient interface through a conduit or tube in an air circuit that fluidly connects to the anterior side of the patient interface so that, when the patient interface is positioned on the patient's face during use, the conduit extends out of the patient interface forwards away from the patient's face. Usually the conduit is not connected to the patient interface at any other location so the conduit hangs vertically downwards from the interface under gravity. This type of interface may sometimes be referred to as a “tube down” or an “elephant trunk” style of interface.

### 1.2.3.1.4 Pressurised Air Conduit Used for Positioning/Stabilising the Seal-Forming Structure

An alternative type of treatment system comprises a patient interface in which a tube or substantially hollow elongate structure that delivers pressurised air to the patient's airways also functions as part of the structure that positions and stabilises the seal-forming portion of the patient interface to the appropriate part of the patient's face, e.g. the headgear. This means that the headgear forms part of the air circuit. For the purposes of this specification the terms “tube” and “conduit” should be considered to have the same meaning, unless the context clearly indicates otherwise.

This type of patient interface may be referred to as incorporating ‘headgear tubing’ or ‘conduit headgear’, these terms being understood to be interchangeable for the purposes of this specification unless the context indicates otherwise. Such patient interfaces allow the conduit in the air circuit providing the flow of pressurised air from a respiratory pressure therapy device to connect to the patient interface in a position other than in front of the patient's face. One example of such a treatment system is disclosed in US Patent Publication No. 2007/0246043, the contents of which are incorporated herein by reference, in which the conduit connects to the patient interface through a port positioned in use on top of the patient's head. This may be referred to as a “tube up” configuration.

The Philips DreamWear™ mask includes such conduit headgear/headgear tubing. The length of the DreamWear™ headgear tubes cannot be adjusted. Consequently, the DreamWear™ headgear is supplied in three different sizes to cater for different sized patient faces. Providing a greater number of different sizes may increase the complexity and cost to manufacture the headgear and may result in larger packaging. Additionally, a supply of discretely sized masks may limit the extent to which differently sized patient heads can be accommodated. There may be a greater chance of some patients being unable to achieve what they consider a comfortable fit if forced to choose between discrete sizes that are not adjustable in length.

Some patients may prefer interfaces incorporating headgear tubing as this allows the selection of a tube up configuration, avoiding a conduit connecting to the patient interface at the front of a patient's face. This can be considered to be unsightly and obtrusive. However, other patients may not find the “tube down” style of interface to be an issue.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators for medical applications have particular requirements not fulfilled by more generalised air pressure generators, such as the acoustic noise, reliability, size and weight requirements of medical devices. However, even devices designed for medical treatment may suffer from shortcomings, pertaining to one or more of: comfort, noise, ease of use, efficacy, size, weight, manufacturability, cost, and reliability.

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

The air circuit may engage with a patient interface and/or seal forming structure or alternatively, when conduit headgear is being used, engage with a connection port provided to, or in fluid communication with, the conduit headgear.

Some examples of air circuits are formed from a single length of tubing. This may lead to excessive “tube drag” in which, as the patient turns their head while wearing the patient interface, the weight of the air circuit or its drag as it moves across nearby surfaces affects the tension applied by the positioning and stabilizing structure to the seal forming structure of the patient interface. This may cause the seal forming structure to fully or partially come away from the patient's face and compromise the effect of the flow of pressurised air from the RPT device and the patient interface.

Having an air circuit of a fixed length of tubing may mean manufacturing air circuits in a variety of lengths to provide the patient with options to suit their personal circumstances; for example, the air circuit may be provided in 0.5, 1.0 and 1.5 metre lengths, the patient selecting their preferred length at the time of being provided with their RPT device. Having to provide this variety in air circuit lengths may be a manufacturing and inventory burden.

### 1.2.3.4 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594. US Patent Application Publication No. US 2009/0050156; US Patent Application Publication No. 2009/0044808.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One aspect of the present technology comprises a patient interface for delivery of a flow of pressurised air to an entrance of a patient's airways.

One aspect of the present technology is directed to a respiratory therapy system, the respiratory therapy system comprising:
a respiratory pressure therapy (RPT) device; and/or
a patient interface for delivery of a flow of pressurised air from the RPT device, via an air circuit, to an entrance of a patient's airways, the patient interface comprising:
   a plenum chamber;
   a seal-forming structure provided to the plenum chamber;
   at least one connection port configured to receive the flow of air from the air circuit and to deliver the flow of air to the entrance of the patient's airways via the seal-forming structure;
   one or more positioning and stabilising structures configured to provide a force to hold the seal-forming structure in a therapeutically effective position on a patient's head in use;
      wherein the respiratory therapy system also comprises an air circuit, wherein the air circuit comprises two or more lengths of tubing, wherein each length of tubing has an upstream end and a downstream end, and wherein each length of tubing includes one or more electrical wire(s); and
a connecting mechanism, wherein the connecting mechanism is configured to connect a downstream end of one of the two or more lengths of tubing fluidly and electrically to an upstream end of another of the two or more lengths of tubing.

Another aspect of the present technology is directed to an air circuit for use with a patient interface receiving a flow of pressurised air from the air circuit, the air circuit comprising:
two or more lengths of tubing, wherein each length of tubing has an upstream end and a downstream end, and wherein each length of tubing includes one or more electrical wire(s); and
a connecting mechanism, wherein the connecting mechanism is configured to connect a downstream end of one of the two or more lengths of tubing fluidly and electrically to an upstream end of another of the two or more lengths of tubing.

In examples, the connecting mechanism comprises a first cuff and a first collar for a downstream end of a first length of tubing and a second cuff and a second collar for an upstream end of a second length of tubing.

In examples, the first cuff and the second cuff are secured to their respective lengths of tubing with an overmould.

In examples, the first cuff includes a mouth, an inner surface of the mouth forming part of a flow path through the first length of tubing.

In this example, the mouth of the first cuff includes a lip.

In this example, the lip of the mouth of the first cuff circumscribes the mouth.

In examples, the first cuff is configured with at least one electrical contact that is electrically connected to the one or more electrical wire(s) of the first length of tubing.

In this example, the first cuff is configured with one electrical contact for each of the one or more electrical wire(s) of the first length of tubing.

In examples, the at least one electrical contacts of the first cuff extend forward of the mouth of the first cuff.

In examples, the electrical connection between the at least one electrical contact of the first cuff and the electrical wire(s) of the first length of tubing comprises at least one conductor path.

In examples, the second cuff is configured with a contact surface that, in use, is received or otherwise contacted by the electrical contact of the first cuff.

In examples, the second cuff includes a mouth, an inner surface of the mouth forming part of a flow path through the second length of tubing.

In examples, the mouth of the second cuff includes a lip complementary to the lip of the first cuff.

In this example, the lip of the mouth of the second cuff circumscribes the mouth.

In examples, the second cuff is configured with a contact surface for each of the electrical contacts of the first cuff.

In this example, the contact surface of the second cuff is provided to an outer surface of the cuff.

In examples, the contact surface of the second cuff is electrically connected to at least one or more electrical wire(s) of the second length of tubing.

In examples, the electrical connection between the contact surface of the second cuff and the electrical wire(s) of the second length of tubing comprises at least one conductor path.

In examples, the first collar is configured to fit over the first cuff.

In examples, the first collar comprises a first end and a second end.

In examples, the first end of the first collar is configured to engage with the overmould and/or an exterior surface of the first cuff.

In examples, the second end of the first collar is configured with a mouth.

In examples, the mouth of the first collar, in use, extends forward of the mouth of the first cuff.

In this example, the mouth of the first collar has an interior surface, wherein the interior surface is configured with one or more recesses for an exterior surface of the electrical contact of the first cuff.

In examples, the first collar is configured with an interlocking feature for engagement with a complementary interlocking feature provided to the second collar.

In examples, the interlocking feature of the first collar comprises one or more keys arranged around an exterior surface of the mouth.

In examples, the second collar is configured to fit over the second cuff.

In examples, the second collar comprises a first end and a second end.

In examples, the first end of the second collar is configured to engage with the overmould and/or an exterior surface of the second cuff.

In examples, the second collar is configured with a mouth, the mouth being defined, in use, by an inner surface of the collar and an outer surface of the second cuff.

In examples, the mouth of the second collar is configured to receive the mouth of the first collar.

In examples, the mouth of the second cuff extends forward of the mouth of the second collar.

In other examples, the mouth of the second collar extends forward of the second cuff.

In examples, the complementary interlocking feature of the second collar is one or more channels configured to receive the one or more keys of the first collar.

In examples, the channels of the second collar include a first leg and a second leg, wherein the second leg is perpendicular to the first leg.

In this example, the one or more keys of the first collar are configured to be only moveable along the first leg of the one or more channels of the second collar in a linear movement.

In this example, the one of more keys of the first collar are configured to be only moveable along the second leg of the one or more channels of the second collar in a rotational movement.

In this example, when the one or more keys of the first collar are engaged with the first leg of the one or more channels of the second, the first length of tubing and second length of tubing are connected, but not locked.

In this example, when the one or more keys of the first collar are engaged with the second leg of the one or more channels of the second collar, the first length of tubing and second length of tubing are connected and locked.

In examples, an exterior surface of the first collar comprises a first gripping surface and an exterior surface of the second collar comprises a second gripping surface.

In this example, the first gripping surface and second gripping surface are arranged to be continuous in a first condition and discontinuous in a second condition.

In this example, the first condition is when the first length of tubing and second length of tubing are connected, but not locked.

In this example, the second condition is when the first length of tubing and second length of tubing are connected and locked.

In examples, the downstream end of the second length of tubing may connect to one of the at least one connection port of the patient interface or an upstream end of a third length of tubing.

In other examples, the downstream end of the second length of tubing may connect to a sensor module that is upstream from the patient interface.

In examples a downstream end of the air circuit connects to the at least one connection port.

In examples, the connection port to which the air circuit is connected is provided to an anterior side of the plenum chamber.

In examples, the connection port to which the air circuit is connected is provided to the one or more positioning and stabilising structures, wherein the one or more positioning and stabilising structures is configured with a flow path for pressurised air to the patient interface.

In some examples, the patient interface and/or one or more positioning and stabilising structures may be configured with two or more connection ports. The connection port not being used may be sealed with a stop or like.

In examples, the plenum chamber(s) and/or seal-forming structure(s) are configured as one or more of: a nasal mask, nasal cushion, nasal pillows or a full-face mask.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a patient interface that may be washed in a home of a patient, e.g., in soapy water, without requiring specialised cleaning equipment.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF DRAWINGS

The present technology is illustrated by way of example, and not by way of limitation, in the figures of the accompanying drawings, in which like reference numerals refer to similar elements including:

FIG. 1A shows a system including a patient 1000 wearing a patient interface 3000, in the form of nasal pillows, receiving a flow or supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a flow or supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a flow or supply of air at positive pressure from an RPT device 4000. Air from the RPT device is humidified in a humidifier 5000, and passes along an air circuit 4170 to the patient 1000. The patient is sleeping in a side sleeping position.

FIG. 2 shows an air circuit 4170 in accordance with one form of the present technology, the air circuit being formed from at least two lengths of tubing held together with a connecting mechanism.

FIG. 3 shows the ends of the respective lengths of tubing that form the air circuit of FIG. 2.

FIGS. 4A to 4D show the end of one of the respective lengths of tubing of FIG. 3, including a first cuff and collar of the connecting mechanism.

FIGS. 5A to 5C show the end of the other of the respective lengths of tubing of FIG. 3, including a second cuff and collar of the connecting mechanism.

FIGS. 6A to 6C shows the manner of connection of the respective lengths of tubing using the first and second cuffs and collars respectively of the connecting mechanisms of FIGS. 4A to 4D and 5A to 5C.

FIG. 7 shows a cross-sectional view of the connecting mechanism for the two lengths of tubing of the air circuit of FIG. 2.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

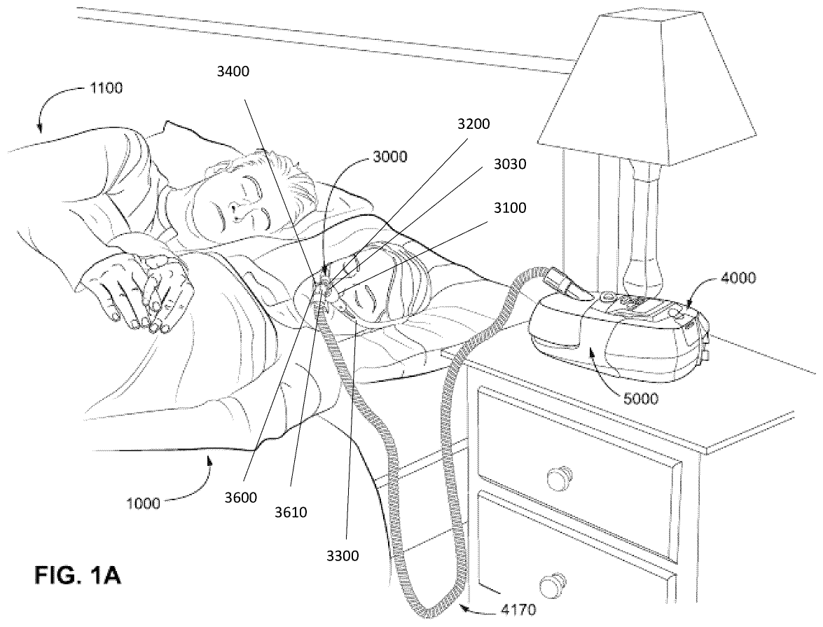

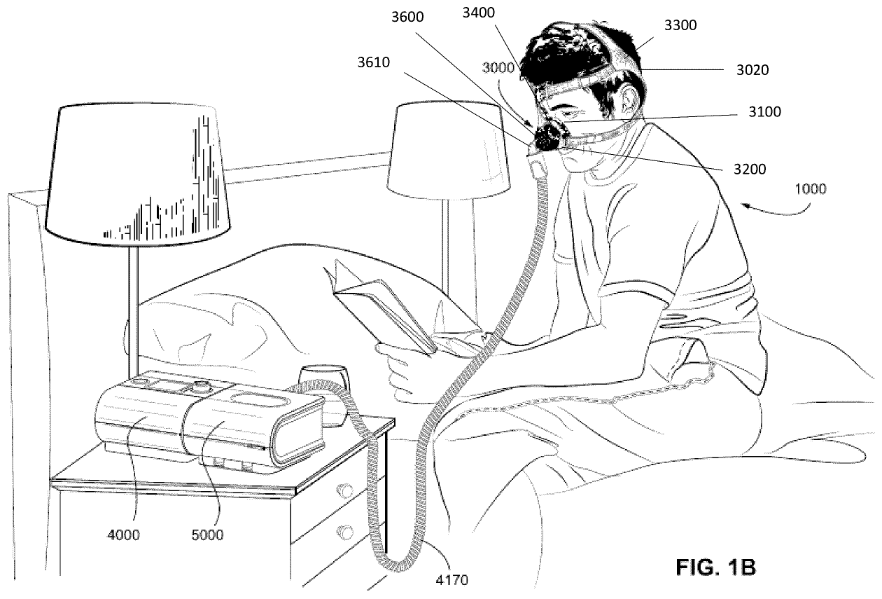

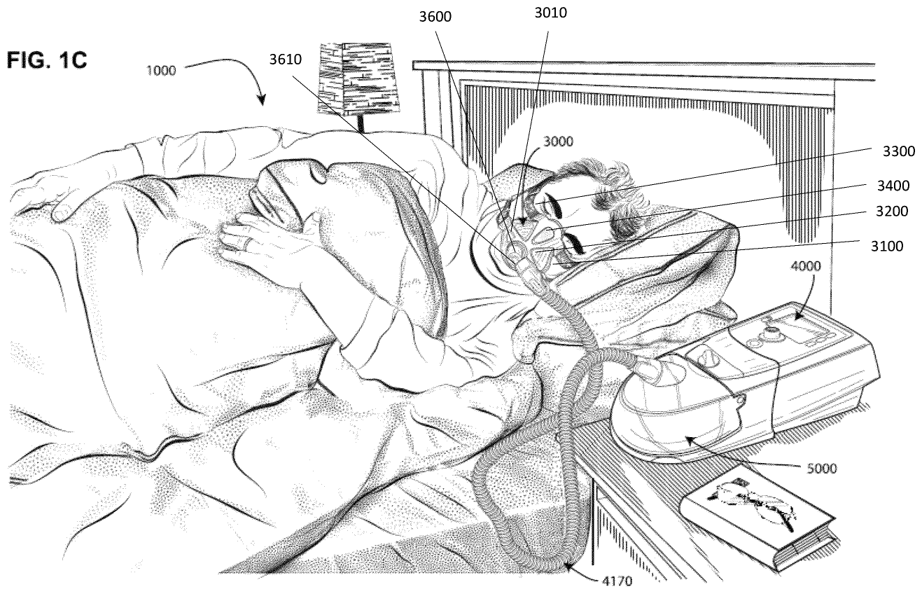

In one form, the present technology comprises a respiratory therapy system for treating a respiratory disorder. As shown in FIGS. 1A to 1C, the respiratory therapy system may comprise an RPT device 4000 for supplying a flow of pressurised air to the patient 1000 via an air circuit 4170 and a patient interface 3000.

### 4.2.1 Air Circuit

The air circuit 4170 comprises the conduit that delivers the flow of pressurised air to the patient interface. It is typically a length of tubing of biocompatible plastics material fluidly connected at an upstream end to the RPT device 4000 and to the patient interface 3000 at a downstream end.

The air circuit 4170 may also include electrical wiring integrated by, for example, over-moulding techniques or the like, into the tubing such that electrical current may be conducted along the length of the tubing.

In one example, the electrical wiring may run about the circumference of the tubing in a manner similar to a coil. In one example, the tubing may be corrugated, to confer a degree of flexibility, with the electrical wiring moulded or otherwise embedded into the corrugations. In another example, the electrical wiring may run axially along the length of the tubing.

In some examples, the electrical wiring forms part of a heating element to warm the flow of pressurised air flowing through the tubing. In some examples, the electrical wiring is part of an electronic communication system for the RPT device 4000 and/or patient interface 3000, transmitting signals from one to the other along the air circuit 4170.

In some examples, the upstream end of the air circuit 4170 is configured as a connector that engages with the RPT device 4000, but in other examples, the upstream end of the air circuit may be configured to engage with a connector which in turn engages with the RPT device. Regardless, this engagement may be by way of snap-lock fittings, complementary threads or similar arrangements.

In some examples, the downstream end of the air circuit 4170 is configured as a connector that engages with the patient interface 3000. This engagement may be by way of snap-lock fittings, complementary threads or similar arrangements.

In each of FIGS. 1A to 1C the air circuit 4170 is shown as a single length of tubing. This allows the patient 1000 to position the RPT device 4000 some distance away if this is their preference or if they are limited by places in which the RPT device 4000 may be placed.

### 4.2.1.1 Connecting Mechanism for Air Circuit

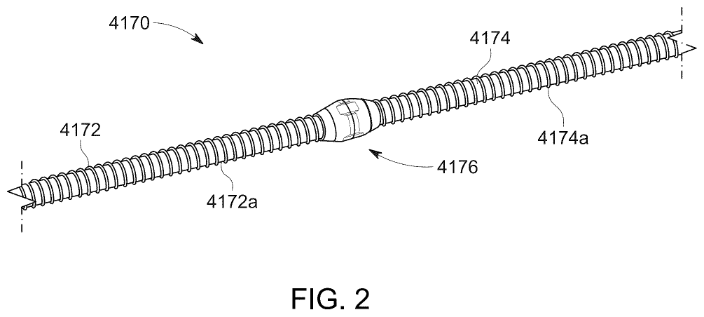

As shown in FIG. 2, the air circuit 4170 may be formed from two or more lengths of tubing, in which the downstream end of a first length of tubing 4172 and the upstream end of a second length of tubing 4174 are held together through the use of a connecting mechanism 4176.

In the illustrated example, the first length of tubing 4172 has an upstream end, which may be configured to be fluidly connected to the RPT device (not shown in FIG. 2 but 4000 in FIGS. 1A to 1C), either directly or via a separate connector, and a downstream end, which is fluidly connected to an upstream end of the second length of tubing 4174. The downstream end of the second length of tubing 4174 is configured to be fluidly connected to the patient interface (not shown in FIG. 2 but 3000 to FIGS. 1A to 1C). In another example, the downstream end of the second length of tubing 4174 may be configured to be fluidly connected to an upstream end of a third length of tubing (not shown). In yet another example, the downstream end of any of the first, second, or any following successive lengths of tubing may be configured to be fluidly connected to another component of the overall respiratory therapy system, including, for example, a sensor module interposed between adjacent lengths of tubing or between the patient interface and the downstream end of the adjacent length of tubing.

The first length of tubing 4172 and second length of tubing may be configured with corrugations 4172a, 4174a, formed as a spiral winding around their respective lengths along the exterior surfaces. Electrical wiring (not visible) may be embedded within the corrugations. The electrical wiring may be a single wire or a plurality of wires.

The connecting mechanism 4176 is configured such that the respective lengths of tubing forming the air circuit 4170 are in communication fluidly, to allow passage of pressurised air from the upstream end of the first length of tubing 4172, which is connected to the RPT device, to the downstream end of the second length of tubing 4174, which is connected to the patient interface (or alternatively another length of tubing). The connecting mechanism is also configured such that the respective lengths of tubing forming the air circuit are in communication electrically, to allow passage of electricity or electronic signals from the upstream end of the air circuit to the downstream end, or vice versa.

Having the air circuit 4170 made up of multiple lengths of tubing allows the patient to adjust the length of the air circuit to their personal preference and potentially avoid or minimise “tube drag” when wearing the patient interface 3000. The connecting mechanism ensures that the two or more separate lengths of tubing comprising the air circuit are in fluid and electrical communication, thus retaining the functionality of an air circuit made up of a single length of tubing.

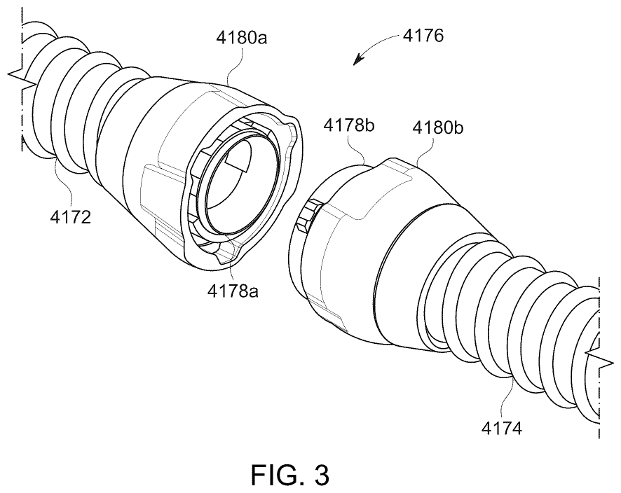

In one example, the connecting mechanism 4176 may comprise a plurality of components as shown in FIG. 3. The respective ends of the lengths of tubing 4172, 4174 are provided with a first cuff 4178a and a second cuff 4178b and a first collar 4180a and a second collar 4180b. The collars pass over and surround their respective cuffs.

The respective cuffs and collars are configured to engage with each other in a “poke-and-yoke” fashion for a simple and straightforward connection, thereby unifying the two lengths of tubing. Other lengths of tubing may be added, so long as the ends to be connected have the necessary connecting mechanism. This makes it easy for a user to form the air circuit 4170 in a length that best matches their personal preference.

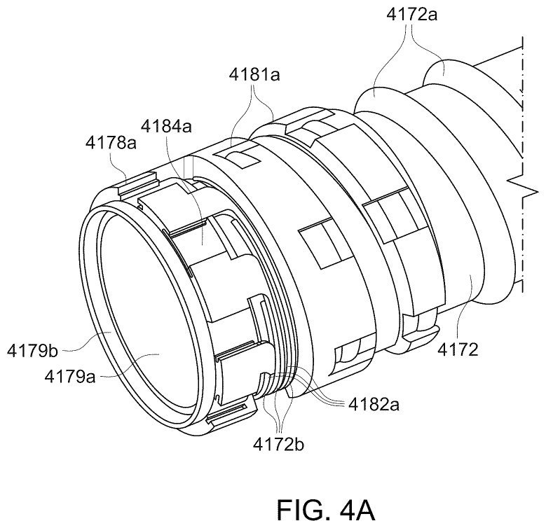

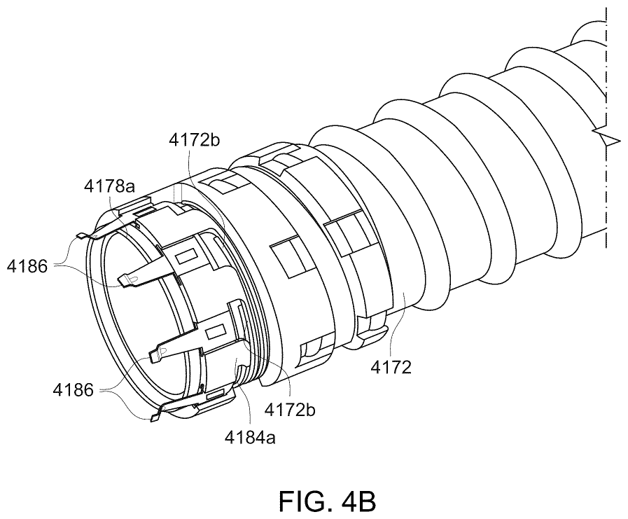

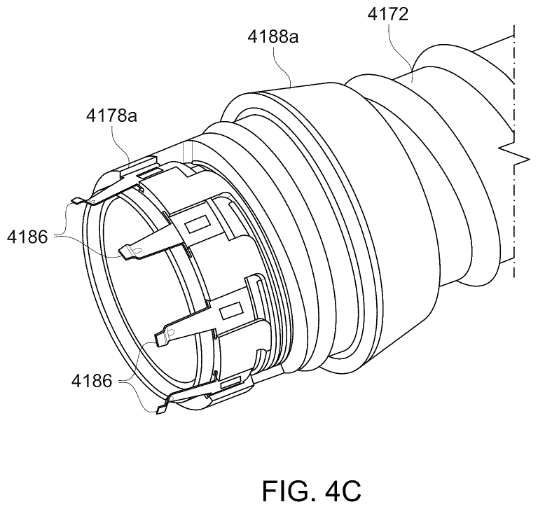

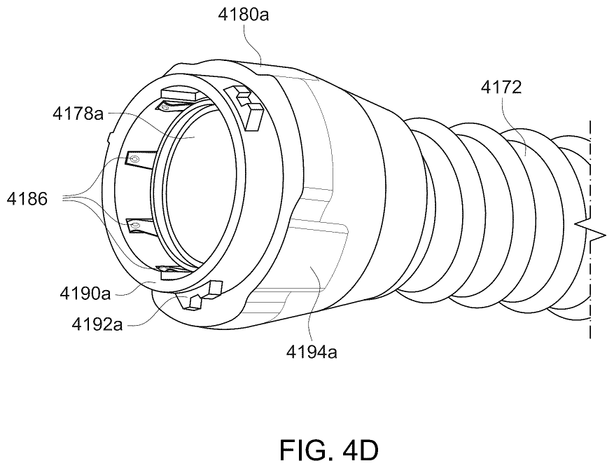

The downstream end of the first length of tubing 4172 is shown in FIGS. 4A to 4D with the first cuff 4178a in place. The first cuff includes a mouth with an interior surface 4179a that may form part of the flow path for pressurised air passing through the first length of tubing. The cuff includes a lip 4179b which circumscribes the mouth.

As best seen in FIG. 4A, the first cuff is a moulding of an appropriate plastics material suitable for use with laser direct structuring (LDS). LDS should be understood to be a technique used for forming a conductor track on three-dimensional components although other techniques used for forming a conductor track or circuit layout may be used instead. As the interior surface 4179a of the first cuff may form part of the flow path for the pressurised air passing through the air circuit 4170, the plastics material from which it is configured should preferably be a biocompatible plastics material. In one example, since it is part of the flow path of pressurised air, the interior surface may include a coat of silicone or silicone elastomer for sake of hygiene. In alternative non-limiting examples, a thermoplastic elastomer (TPE) or resin potting compound may be used.

The first cuff 4178a is configured with structures 4181a that are complementary to corrugations 1472a provided to the first length of the tubing 4172. During assembly of the air circuit, the cuff is threaded onto the corrugations of the tubing, into which electrical wiring 4172b is embedded. In another example, the cuff may be formed from two components which connect together in a snap-lock or encapsulating-type arrangement. The end of the tubing may be placed in one of the two components of the cuff, and the second component is then secured to the first. The exposed ends of the electrical wiring are received in grooves 4182a provided to the first cuff. The electrical wiring may be retained in the grooves through the use of soldering techniques or by overcoating the grooves with adhesive or the like. However, other ways of retaining the electrical wiring within the grooves will be readily envisaged. The grooves of the cuff terminate in a well 4184a in which a conductor path or track formed using LDS is disposed. The ends of the electrical wiring are in contact with the conductor path of the well. The conductor path of the well may also extend to the grooves 4182a.

In some examples, the conductor path or track may include or otherwise incorporate electrical circuits for ancillary electrical/electronic components that may be part of the respiratory therapy system. For example, the interior surface of the cuff may be configured with means for attaching optional sensing equipment. In one such example, a portion of the inner surface may be configured with contact or connection points with an electrical circuit laid down by LDS or similar techniques to which sensing equipment may be fitted. In an example, the connection point may be a protrusion extending radially inwards; such a configuration may allow a sensor to be placed into the flow path of the length of tubing. Examples of other ancillary electrical/electronic components that may be used include reed switches, resistors or capacitors or such like.

In FIG. 4B, the first cuff 4178a is shown with contacts 4186 which may be added to the well 4184a through the use of appropriate fastening or securing techniques, such as soldering or the like. These contacts, which contact the conductor path, are formed from a sprung metal that is electrically conductive. The number of contacts provided, four in the illustrated example, correspond to the number of individual wires 4172b. In the illustrated example, the contacts 4186 extend forward, beyond the end of the first cuff, and are relatively exposed. However, in other examples, the ends of the contacts may terminate within the end of the first cuff. In FIG. 4C, the first cuff 4178a is secured to the first length of tubing 4172 through an over-mould 4188a. This secures the first cuff relative to the tubing and seals the respective surfaces.

In FIGS. 4B and 4C, it will be seen that the contacts 4186 of the first cuff 4178a are exposed. In FIG. 4D, a first collar 4180a is provided to cover the contacts and the mouth 4179b of the first cuff, passing over the first cuff 4178a such that a first end abuts the over-mould 4188a. In one example, the first end of the first collar may be permanently fixed in place relative to the first cuff and/or first length of tubing via a snap-lock arrangement with a portion of the exterior surface of the first cuff and/or over-mould. In another example, the manner of connecting the first collar to the first cuff is such that that it could be made removable to allow access to the well (not visible in this figure but 4184a in FIGS. 4A and 4B) for maintenance purposes (for example to inspect the electrical connection). In one example, the internal surfaces of the first collar may include fittings that engage and key in with complementary fittings provided to the exterior surface of the first cuff. This may provide means for controlling the rotation angle of the first collar in use.

As seen in FIG. 4D, the first collar 4180a is provided with a mouth 4190a at its second end. Around the exterior circumference of this mouth is provided a number of interlocking features in the form of keys 4192a. In this example, three keys are shown, arranged equi-distance around the exterior circumference of the mouth. However, there may be more, or less, depending on manufacturing and/or user preferences.

The second end of the first collar 4180a extends forward of the mouth of the cuff 4178a, covering the contacts 4186. In this arrangement, the second end of the first collar, once in place, pre-loads the contacts, placing them under tension and urging them, in use, into a position appropriate for an optimal electrical connection with the mating surfaces of the second cuff. The interior circumference of the mouth is provided with recesses (obscured) into which the contacts 4186 of the cuff are disposed once the first collar is fitted to the first length of tubing 4172.

A portion of the outer surface of the first collar 4180a is contoured to provide a gripping surface 4194a for the user.

As the first collar 4180a (and the second collar 4180b) may be relatively exposed to wear and tear as the user handles the connecting mechanism 4176, these may be formed from a durable plastics material. In one example, the plastics material may be a polycarbonate although it will be appreciated that other suitable plastics material may be used.

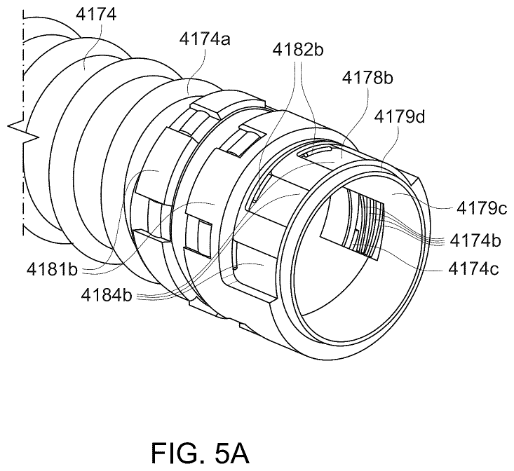

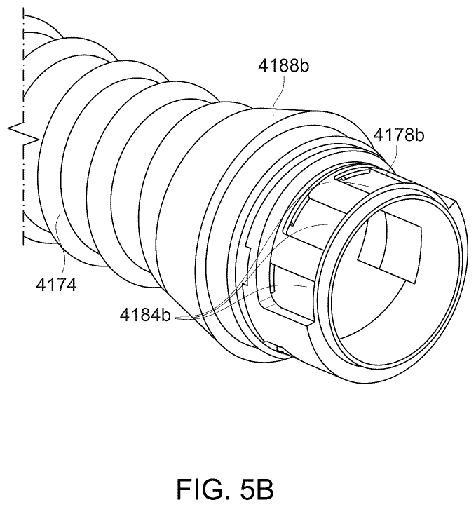

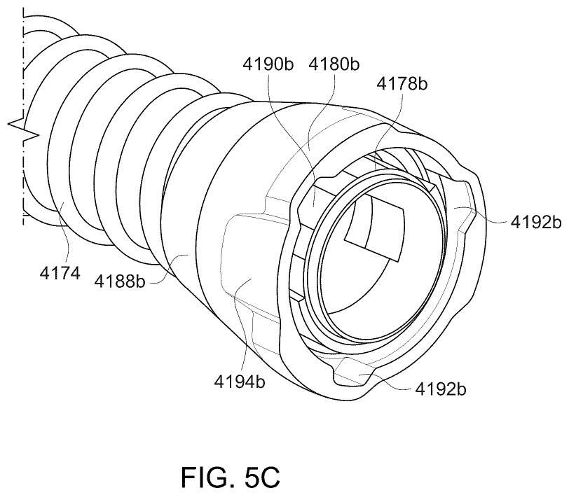

The upstream end of the second length of tubing 4174 is shown in FIGS. 5A to 5C and its manner of construction may be largely similar to that described for the first length of tubing. The second cuff 4178b is mated to the end of the tube which has electrical wiring 4174b (four individual wires are seen here, corresponding to the four contacts of the first length of tubing) embedded within the corrugations 4174a.

In FIG. 5A, it will be seen that a cut edge 4174c of the tubing 4174 exposes the ends of the electrical wiring 4174b. These are received in grooves 4182b that are communicative with a pad 4184b that provides a conductor path, formed using LDS techniques or similar, in use. It will be understood that in use, when the respective first and second lengths of tubing are connected, the contacts 4186 of the first length of tubing touch the pad 4184b of the second length of tubing. An over-mould 4188b secures and seals the cuff 4178b, including its cut edge, to the second length of tubing 4174 as seen in FIG. 5B.

The mouth of the second cuff 4178b may be configured with a lip 4179d about its external surface. In use, this mates with the lip 4179b of the first cuff 4178a and helps facilitate a connection between the respective lengths of tubing 4172, 4174. Similar to the first cuff, in some examples, the interior surface 4179c of the second cuff may be coated with a silicone or silicone elastomer for hygiene reasons. In alternative non-limiting examples, a TPE or resin potting compound may be used.

Finally, a second collar 4180b is provided to the upstream end of the second length of tubing 4174 of FIG. 5C, fitting over and being secured to the second cuff 4178b. In one example, the internal surfaces of the second collar may include fittings that engage and key in with complementary fittings provided to the exterior surface of the second cuff. This may provide means for controlling the rotation angle of the second collar in use. The second collar 4180b is configured with at least a portion of its exterior surface contoured to provide a gripping surface 4194b. A mouth 4190b is defined between the interior surface of the second collar and the exterior surface of the second cuff 4178b. In use, this receives the mouth 4190a of the first collar 4180a provided to the first length of tubing 4172.

Three interlocking features in the form of channels 4192b are shown, arranged equi-distance around the interior circumference of the mouth 4190b of the second collar 4180b. These channels have a first leg (the entrance to which is what is seen in FIG. 5C), and although not visible, a second leg substantially at right angles to the first leg. It will be appreciated that these channels engage with the keys 4192a provided to the mouth 4190a of the collar 4180a of the first length of tubing 4172 shown in FIG. 4D. Other variations to the interlocking features will be readily envisaged; in one example, the arrangement may be reversed such that the second collar is provided with keys that engage with channels provided to the first collar. In a further example, the interlocking features may be an entirely different physical configuration.

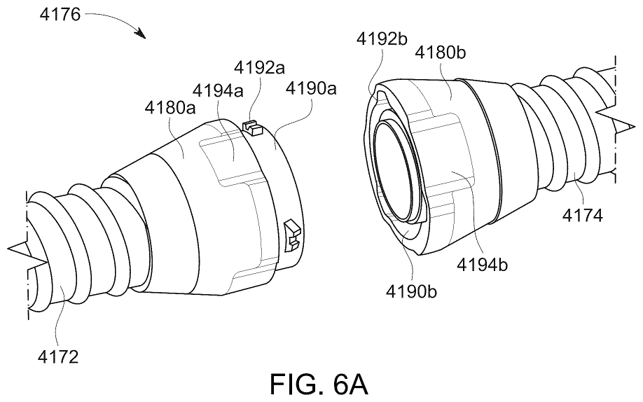

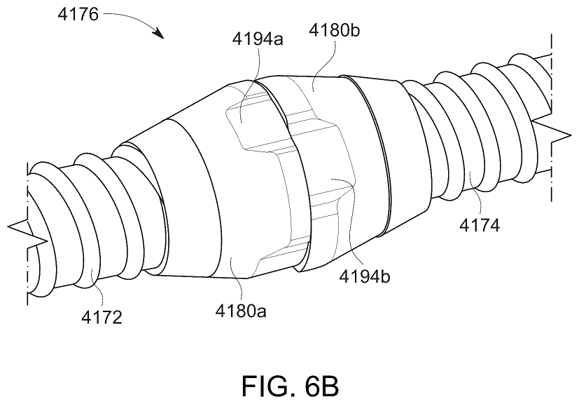

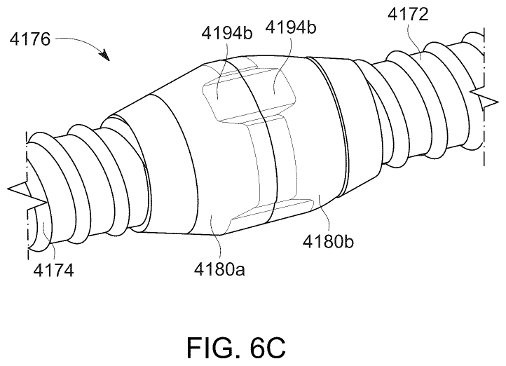

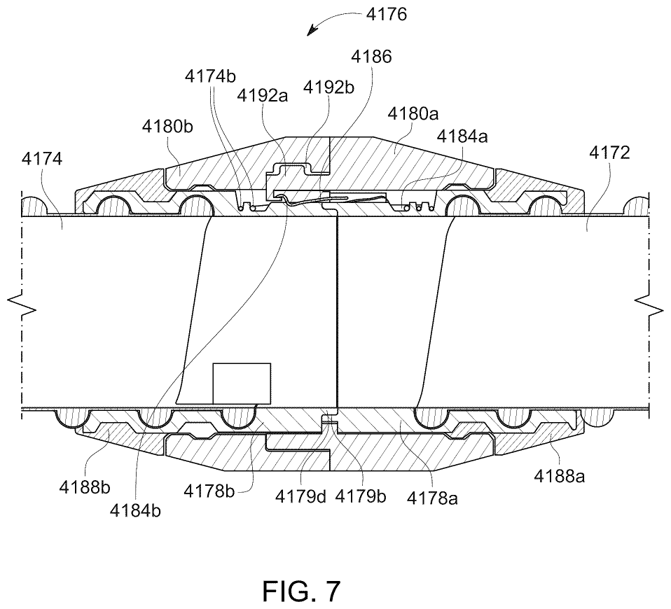

The manner of operating the connecting mechanism 4176 to unify the respective first length of tubing 4172 and second length of tubing 4174 is shown in FIGS. 6A to 6C and FIG. 7.

First, as shown in FIG. 6A, the downstream end of the first length of tubing 4172 and the first collar 4180a is presented to the upstream end of the second length of tubing 4174 and the second collar 4180b. The mouths 4190a, 4190b and their respective keys 4192a and channels 4192b are in alignment; this facilitates a “poke-and-yoke” arrangement such that it is physically impossible to incorrectly connect the first length and second length of tubing. This greatly simplifies ease of use of the overall connecting mechanism 4176.

When the keys 4192a and channels 4192b are in alignment, the respective collars 4180a, 4180b may be advanced towards each other. Thus the collars 4180a, 4180b are connected to each other in FIG. 6B, i.e. in a first condition, but have yet to be locked in place, i.e. put into a second condition. The keys 4192a of the first collar 4180a of the first length of tubing 4172 are engaged with the first leg of the channels 4192b of the second collar 4180b of the second length of tubing 4174. It will be noted that the gripping surfaces 4194a, 4194b, of the respective collars are discontinuous, i.e. not in alignment. This may serve as an indicator to the user that although the respective lengths of tubing 4172, 4174 are in the first condition, they have yet to be put into the second condition.

As such, the gripping surfaces 4194a, 4194b remain out of alignment until one of the collars 4180a, 4180b is axially rotated relative to the other in a twisting action into the position shown in FIG. 6C. This urges the keys 4192a along the second leg of the channels 4192b. In the illustrated example, the length of the second leg of the channels is such that the user's twisting action is around 40° of rotational movement. However, the length of the second leg may be longer (or shorter) if desired for ease of manufacturing and/or use.

In some examples, the second channel may be provided with a notch or similar recess at its end. A biasing member, such as a spring (not shown) may be present at the base of the mouth 4190b of the collar 4180b of the second length of tubing 4174 such that in use, this urges the key 4192a of the collar 4180a of the first length of tubing 4172 into the notch. This may provide greater certainty for the user of the engagement of the locking mechanism 4176.

As best seen in FIG. 7, the respective keys 4192a and channels 4192b interlock to secure the first length of tubing 4172 to the second length of tubing 4174. An axial rotation in a reverse direction is required to disengage the respective lengths of tubing. There is also an overlap between the respective lips 4179b, 4179d of the first and second cuffs, 4178a, 4178b. In the illustrated example, the lip 4179d of the second cuff 4178b extends slightly forward of the mouth of the second collar 4180b. However, in other examples, the lip 4179d may be slightly rearward, or in the same plane, of the mouth of the second collar 4180b.

As discussed in respect of FIG. 5C, the channel 4192b of the collar 4180b of the second length of tubing has a first leg with a second leg at right angles to the first leg although the second leg was not visible in that figure.

In FIG. 7 the second leg of the channel 4192b of the collar 4180b of the second length of tubing 4174 can be seen with the key 4192a of the collar 4180a of the first length of tubing 4172 in place, locking the respective lengths of tubing together. The user can confirm the locked position by the alignment of the gripping portions of the respective collars.

The easy-to-operate connecting mechanism 4176 allows the formation of an air circuit 4170 for a respiratory therapy device 4000 which is fluidly communicative with a patient interface 3000, through the linking of a first length of tubing 4172 to a second length of tubing 4174. It is also electrically communicative by virtue of the contacts 4186 of the cuff 4178a of the first length of tubing 4172 touching the pad 4184a which has a conductor path to the wires 4174b.

The patient 1000 has the ability to customise the length of the air circuit 4170 to their preferences/circumstances of use; for example, when seated next to a table or the like, a shorter air circuit may be preferred but when sleeping, a longer air circuit may be required. Additionally, the connecting mechanism is configured to be relatively low-profile and unobtrusive, potentially minimising “tube drag” as the air circuit 4170 moves across furniture or bedding in response to movement by the patient or their sleeping partner 1100.

### 4.2.2 Patient Interface

A non-invasive patient interface 3000 in accordance with one aspect of the present technology comprises the following functional aspects: a seal-forming structure 3100, a plenum chamber 3200, a positioning and stabilising structure 3300, a connection port 3600 provided to the anterior side of the plenum chamber for connection to the downstream end of the air circuit 4170.

In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH₂O with respect to ambient. In another form of the present technology, the patient interface is constructed and arranged to be able to provide a supply of air at a positive pressure of at least 10 cmH₂O with respect to ambient. In yet another form of the present technology, the patient interface 3000 is constructed and arranged to be able to provide a supply of air at a positive pressure of at least 20 cmH₂O with respect to ambient.

### 4.2.3 Seal-Forming Structure

In one form of the present technology, the patient interface includes a seal-forming structure 3100 that provides a target seal-forming region and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a soft, flexible, resilient material and/or biocompatible material, e.g. silicone rubber.

In certain forms of the present technology, a system is provided comprising more than one a seal-forming structure 3100, each being configured to correspond to a different size and/or shape range. For example, the system may comprise one form of a seal-forming structure 3100 suitable for a large sized head, but not a small sized head and another suitable for a small sized head, but not a large sized head.

### 4.2.3.1 Sealing Mechanisms

In one form, the seal-forming structure includes a sealing flange (not visible in FIGS. 1A to C) utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.2.4 Plenum Chamber

The patient interface 3000 includes a plenum chamber 3200 that has a perimeter that is shaped to be complementary to the surface contour of the face of an average person in the region where a seal will form in use. In one form of the present technology, the plenum chamber may be part of a full-face mask, ora-nasal, nasal mask, nasal pillows or nasal cushion.

In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

The plenum chamber has an anterior side, which should be understood to be the external surface of the plenum chamber facing away from the patient in use. In some forms of the present technology, the plenum chamber may be comprised of two or more components. In one example, the anterior surface may comprise part of a rigid shell to which other portions of the plenum chamber are connected or mounted, either permanently, through the use of appropriate bonding or over-moulding techniques, or temporarily, through the use of complementary or snap lock fasteners.

In some examples, the plenum chamber and/or seal-forming structure may be configured to be removed from the patient interface and replaced with another plenum chamber and/or seal-forming structure, for example ones that differ structurally in some way, for examples ones of different size, type or shape. In this way, the patient may, for example, swap between a plenum chamber and seal-forming structure configured as a nasal mask, a full-face mask, a nasal cushion, and nasal pillows, or between small, medium and large masks of the same type. The plenum chamber and seal-forming structure may form a sub-assembly or module to make such interchanging more convenient for a patient.

The plenum chamber has a posterior side, which should be understood to be the internal surface of the plenum chamber. The posterior side of the plenum chamber has provided to it the seal-forming structure which, in use, receives the nose and/or mouth of the patient.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface and help improve compliance with therapy.

### 4.2.5 Positioning and Stabilising Structure

The seal-forming structure 3100 of the patient interface 3000 of the present technology may be held in a sealing position when being worn by the patient through the use of the positioning and stabilising structure 3300. Positioning and stabilising structure 3300 may be referred to as “headgear” since it contacts and engages with the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

### 4.2.6 Vent Structure

In one form, the patient interface 3000 includes a vent structure 3400, shown in FIGS. 1B and 1C, which should be understood to be the main vent structure, constructed and arranged to allow for the washout of exhaled gases, e.g. carbon dioxide.

In certain forms the vent structure 3400 is configured to allow a continuous vent flow from an interior of the plenum chamber 3200 to ambient whilst the pressure within the plenum chamber is positive with respect to ambient. The vent structure 3400 is configured such that the vent flow rate has a magnitude sufficient to reduce rebreathing of exhaled CO₂ by the patient while maintaining the therapeutic pressure in the plenum chamber in use.

One form of the vent structure 3400 in accordance with the present technology comprises a plurality of holes, for example, about 20 to about 80 holes, or about 40 to about 60 holes, or about 45 to about 55 holes.

In one form of the technology the vent structure 3400 may be configured to be an integral part of the plenum chamber and/or the seal-forming structure. In a further form, in which the positioning and stabilising structure is configured, at least partially, with tubes that allow pressurised air to flow from a connection port separate to the plenum chamber, the vent structure may be an integral part of the positioning and stabilising structure. In other forms of the technology the vent structure 3400 may be a separate component to the plenum chamber, seal-forming structure, connection port and/or positioning and stabilising structure, but may be provided to one of those components. The vent structure may be detachable for cleaning and replacing. In some forms of the present technology, the patient interface may be provided with more than one vent structure.

In one form of the technology the vent structure 3400 is formed from rigid medical grade plastics material. In alternative forms, the vent structure is formed from a soft woven mesh. In these forms, the mesh may be bounded by a rigid or semi-rigid frame to confer some structural integrity to the vent structure. In other forms, it may be formed as a moulding from a rigid plastics material.

### 4.2.7 Connection Port

The connection port 3600 allow for connection of the patient interface 3000 to the downstream end of the air circuit 4170.

In some examples, such as that shown in FIGS. 1A and 1C, the connection port 3600 may comprise an elbow received in a fluid connection opening of the patient interface 3000. The elbow may be received in a ring in the fluid connection opening and may be configured to swivel within the ring. The fluid connection opening may be also considered a connection port itself.

### 5 GLOSSARY

For the purposes of the present technology disclosure, in certain forms of the present technology, one or more of the following definitions may apply. In other forms of the present technology, alternative definitions may apply.

### 5.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

Respiratory Pressure Therapy (RPT): The application of a supply of air to an entrance to the airways at a treatment pressure that is typically positive with respect to atmosphere.

Ventilator A mechanical device that provides pressure support to a patient to perform some or all of the work of breathing.

### 5.2 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker.

Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.3 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).

‘Soft’ materials may include silicone or thermo-plastic elastomer (TPE), and may, e.g. readily deform under finger pressure.

‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.4 Anatomy of the Face

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Nares (Nostrils): Approximately ellipsoidal apertures forming the entrance to the nasal cavity. The singular form of nares is naris (nostril). The nares are separated by the nasal septum.

### 5.5 Patient Interface

Anti-asphyxia valve (AAV): The component or sub-assembly of a mask system that, by opening to atmosphere in a failsafe manner, reduces the risk of excessive CO₂ rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft flexible, elastic material such as a laminated composite of foam and fabric. Alternatively, the headgear may be in the form of one or two gas delivery tubes which may conform somewhat to the shape of the head.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 6 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may, be conducted concurrently or even synchronously.

It is therefore to be understood that numerous modifications may be made to the illustrative examples and that other arrangements may be devised without departing from the spirit and scope of the technology.

7 REFERENCE SIGNS LIST

| | |
|---|---|
| 1000 | Patient |
| 1100 | Patient's sleeping partner |
| 3000 | Patient interface |
| 3010 | Ora-nasal mask |
| 3020 | Nasal cushion |
| 3030 | Nasal pillows |
| 3100 | Sealing or seal-forming structure |
| 3200 | Plenum chamber |
| 3300 | Positioning and stabilising structure |
| 3400 | Vent structure |
| 3600 | Connection port |
| 3610 | Elbow |
| 4000 | RPT device |
| 4170 | Air circuit |
| 4172 | First length of tubing |
| 4172a | Corrugations (of first length of tubing) |
| 4172b | Electric wires (of first length of tubing) |
| 4174 | Second length of tubing |
| 4174a | Corrugations (of second length of tubing) |
| 4174b | Electric wires (of second length of tubing) |
| 4176 | Connecting mechanism |
| 4178a | First cuff (of connecting mechanism) |
| 4178b | Second cuff (of connecting mechanism) |
| 4179a | Interior surface of mouth (of first cuff) |
| 4179b | Lip of mouth (of first cuff) |
| 4179c | Interior surface of mouth (of second cuff) |
| 4179d | Lip of mouth (of second cuff) |
| 4180a | First collar (of connecting mechanism) |
| 4180b | Second collar (of connecting mechanism) |
| 4181a | Corrugations (of first cuff) |
| 4181b | Corrugations (of second cuff) |
| 4182a | Grooves (in first cuff, for electric wires) |
| 4182b | Grooves (in second cuff, for electric wires) |
| 4184a | Well (in first cuff, for conductor path) |
| 4184b | Contact surfaces (of second cuff) |
| 4186 | Electric contacts (of first cuff) |
| 4188a | Over-mould (of first length of tubing) |
| 4188b | Over-mould (of second length of tubing) |
| 4190a | Mouth (of first collar) |
| 4190b | Mouth (of second collar) |
| 4192a | Key (of first collar) |
| 4192b | Channels (of second collar) |
| 4194a | Gripping surface (of first collar) |
| 4194b | Gripping surface (of second collar) |
| 5000 | Humidifier |

1. A respiratory therapy system, the respiratory therapy system comprising:
a respiratory pressure therapy (RPT) device; and/or
a patient interface for delivery of a flow of pressurised air from the RPT device, via an air circuit, to an entrance of a patient's airways, the patient interface comprising:
a plenum chamber;
a seal-forming structure provided to the plenum chamber,
at least one connection port configured to receive the flow of air from the air circuit and to deliver the flow of air to the entrance of the patient's airways via the seal-forming structure;
one or more positioning and stabilising structures configured to provide a force to hold the seal-forming structure in a therapeutically effective position on a patient's head in use;
wherein the respiratory therapy system also comprises an air circuit, the air circuit comprising:
two or more lengths of tubing, wherein each length of tubing has an upstream end and a downstream end, and wherein each length of tubing includes one or more electrical wire(s); and
a connecting mechanism, wherein the connecting mechanism is configured to connect a downstream end of one of the two or more lengths of tubing fluidly and electrically to an upstream end of another of the two or more lengths of tubing.

2. The respiratory therapy system of claim 1, wherein the patient interface is one of the following mask types: nasal cushion, nasal pillows, ora-nasal mask, or a full-face mask.

3. An air circuit for use in a respiratory therapy system, wherein the air circuit is configured to be connected to a respiratory pressure therapy device at a first end and connected to a patient interface at a second end, the air circuit comprising:
two or more lengths of tubing, wherein each length of tubing has an upstream end and a downstream end, and wherein each length of tubing includes one or more electrical wire(s); and
a connecting mechanism, wherein the connecting mechanism is configured to connect a downstream end of one of the two or more lengths of tubing fluidly and electrically to an upstream end of another of the two or more lengths of tubing.

4. The air circuit of claim 3, wherein the connecting mechanism comprises a first cuff and a first collar for a downstream end of a first length of tubing and a second cuff and a second collar for an upstream end of a second length of tubing.

5. The air circuit of claim 4, wherein the first cuff includes a mouth, an inner surface of the mouth forming part of a flow path through the first length of tubing.

6. The air circuit of claim 4, wherein the second cuff includes a mouth, an inner surface of the mouth forming part of a flow path through the second length of tubing.

7. The air circuit of claim 4, wherein the first cuff is configured with at least one electrical contact that is electrically connected to the one or more electrical wire(s) of the first length of tubing.

8. The air circuit of claim 7, wherein the first cuff is configured with one electrical contact for each of the one or more electrical wire(s) of the first length of tubing.

9. The air circuit of claim 7, wherein the electrical connection between the at least one electrical contact of the first cuff and the electrical wire(s) of the first length of tubing comprises at least one conductor path.

10. The air circuit of claim 7, wherein the second cuff is configured with a contact surface that, in use, is received or otherwise contacted by the electrical contact of the first cuff.

11. The air circuit of claim 10, wherein the contact surface of the second cuff is electrically connected to at least one or more electrical wire(s) of the second length of tubing.

12. The air circuit claim 4, wherein the first collar is configured to fit over the first cuff.

13. The air circuit claim 4, wherein the first collar comprises a first end and a second end.

14. The air circuit of claim 13, wherein the first end of the first collar is configured to engage with an overmould provided to the first length of tubing and/or an exterior surface of the first cuff.

15. The air circuit of claim 13, wherein the second end of the first collar is configured with a mouth.

16. The air circuit claim 4, wherein the second collar is configured to fit over the second cuff.

17. The air circuit claim 4, wherein the first collar is configured with an interlocking feature for engagement with a complementary interlocking feature provided to the second collar.

18. The air circuit of claim 17, wherein the interlocking feature of the first collar comprises one or more keys arranged around an exterior surface of a mouth provided to an end of the first collar.

19. The air circuit of claim 18, wherein the complementary interlocking feature of the second collar is one or more channels configured to receive the one or more keys of the first collar.

20. The air circuit of claim 19, wherein the one or more channels of the second collar include a first leg and a second leg, wherein the second leg is perpendicular to the first leg.

21. The air circuit of claim 20, wherein the one or more keys of the first collar are configured to be only moveable along the first leg of the one or more channels of the second collar in a linear movement.

22. The air circuit of claim 20, wherein the one of more keys of the first collar are configured to be only moveable along the second leg of the one or more channels of the second collar in a rotational movement.

23. The air circuit of claim 4, wherein the second collar is configured with a mouth, the mouth being defined, in use, by an inner surface of the collar and an outer surface of the second cuff.

24. The air circuit of claim 23, wherein a mouth is provided to an end of the first collar and the mouth of the second collar is configured to receive the mouth of the first collar.

25. The air circuit of claim 4, wherein an exterior surface of the first collar comprises a first gripping surface and an exterior surface of the second collar comprises a second gripping surface.

26. The air circuit of claim 25, wherein the first gripping surface and second gripping surface are arranged to be continuous in a first condition and discontinuous in a second condition.

27. The air circuit as claimed in claim 26, wherein the first condition is when the first length of tubing and second length of tubing are connected, but not locked.

28. The air circuit as claimed in claim 26, wherein the second condition is when the first length of tubing and second length of tubing are connected and locked.

29. The air circuit of claim 4, wherein the first cuff and the second cuff are secured to their respective lengths of tubing with an overmould.
